(19) United States
(12) Patent Application Publication
(10) Pub. No.: US 2008/0077143 A1
(43) Pub. Date: Mar. 27, 2008

(54) APPARATUS FOR CONNECTING A LONGITUDINAL MEMBER TO A BONE PORTION

(75) Inventor: ALAN E. SHLUZAS, WEST ROXBURY, MA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: ZIMMER SPINE, INC., MINNEAPOLIS, MN (US)

(21) Appl. No.: 11/861,123

(22) Filed: Sep. 25, 2007

Related U.S. Application Data
(60) Provisional application No. 60/847,330, filed on Sep. 25, 2006.

(51) Int. Cl. A61B 17/56

(52) U.S. Cl. 606/73

(57) ABSTRACT

An apparatus is provided for connecting a longitudinal member to a bone portion. The apparatus includes a fastener engageable with a bone portion, a housing having a first passage configured to receive a longitudinal member and a second passage configured to receive the fastener. The fastener extends through an opening in the housing into the second passage and is movable relative to the housing. The longitudinal axis of the fastener is positionable in any one of a plurality of angular positions relative to a longitudinal axis of the second passage. The apparatus also includes a clamping member configured to be received in threaded engagement within the housing. The clamping member has a first end configured such that when a longitudinal member is positioned in the first passage in the housing, the first end of the clamping member engages the longitudinal member at an angle not orthogonal to an axis along which the clamping member is advanced.

### CROSS REFERENCE TO RELATED APPLICATION

This application claims the benefit of priority to U.S. Provisional Application No. 60/847,330, filed Sep. 25, 2006, the entire disclosure of which is hereby incorporated by reference.

### FIELD OF THE INVENTION

This application relates generally to apparatus for retaining bone portions, and in particular for retaining bones such as vertebrae, in a desired spatial relationship.

### SUMMARY

In one arrangement, the apparatus is configured to provide increased angularity between a fastener and a housing configured to receive a fixation rod, dynamic stabilizer rod, or other longitudinal member. Increased angularity can be provided by providing a first range of angular motion to one side of a longitudinal axis of the fastener and a second range of angular motion to another side of the longitudinal axis of the fastener, the first range of angular motion being greater than the second.

In some embodiments, an apparatus is connectable to a bone portion and includes a longitudinal member, a housing, and a fastener that is engageable with the bone portion. The housing has a passage configured to receive at least a portion of the longitudinal member and an opening through which the fastener is extendable. A clamping mechanism is advanced into engagement with the housing, e.g., along an axis, to clamp the longitudinal member to the housing to restrict or substantially prevent movement of the longitudinal member relative to the housing. The axis along which the clamping mechanism is advanced, which is sometimes referred to herein as a clamping axis, is not orthogonal to the longitudinal member at a location along the longitudinal member closest to the clamping axis, e.g., at a location where the clamping axis intersects the longitudinal member.

In some embodiments, an apparatus connectable to a bone portion includes a fastener, a housing, and a longitudinal member. The fastener is engageable with the bone portion. The housing preferably has an opening through which the fastener is extendable and a passage configured to receive at least a portion of the longitudinal member. The passage can include opposing first and second ends. A clamping mechanism can be advanced into engagement with the housing along an axis. The clamping mechanism is configured to engage the longitudinal member at a first location and a second location. In one technique, the first location is adjacent the first end of the passage and the second location is adjacent the second end of the passage. The first location can be higher than the second location in a direction along the axis. In one technique, a projection of the second location onto the axis is between a projection of the first location onto the axis and a projection of the mid-line of the longitudinal member onto the axis.

In other embodiments, an apparatus connectable to a bone portion includes a fastener, a housing, and a longitudinal member. The fastener is engageable with the bone portion. The housing preferably has a first passage configured to receive at least a portion of the longitudinal member. The housing can include a second passage with a longitudinal axis transverse to the first passage. The fastener extends through an opening in the housing into the second passage in one embodiment. The longitudinal axis of the fastener is positionable in any one of a plurality of angular positions relative to the longitudinal axis of the second passage. A clamping mechanism is advanceable into engagement with the housing along an axis to clamp the longitudinal member to the housing to restrict or substantially prevent movement of the longitudinal member relative to the housing. The axis is not orthogonal to the longitudinal member at a location along the longitudinal member closest to the axis. A spacer preferably is interposed between the fastener and the longitudinal member. The spacer has a top surface that is configured to engage the longitudinal member at an angle that is not orthogonal to the axis.

In other embodiments, a method is provided for using a clamping mechanism to restrict or substantially prevent relative movement between at least two primary members of an apparatus. The apparatus includes a longitudinal member, a fastener engageable with a bone portion, and a housing engageable with the longitudinal member and the fastener. The method comprises advancing or rotating a portion of the clamping mechanism along an axis. The axis is not orthogonal to the longitudinal member at a location along the longitudinal member closest to the axis.

In other embodiments, the method comprises accessing the clamping mechanism through an opening and advancing, e.g., rotating, a portion of the clamping mechanism along an axis. The opening can define a portion of an access path through the skin of the back of the patient to the vertebral site being treated. Such an access path can be formed in a structure or access device. The axis passes through the opening. A plane normal to the longitudinal member at a location along the longitudinal member closest to the axis does not intersect with the opening.

### BRIEF DESCRIPTION OF THE DRAWINGS

Further objects, features and advantages of the invention will become apparent from the following detailed description taken in conjunction with the accompanying figures showing illustrative embodiments of the invention, in which:

FIG. 1 is a perspective view of a portion of one embodiment of an apparatus for connecting a longitudinal member to a bone portion;

FIG. 2 is a plan view of the apparatus of FIG. 1;

FIG. 3 is an end view of the apparatus of FIG. 1;

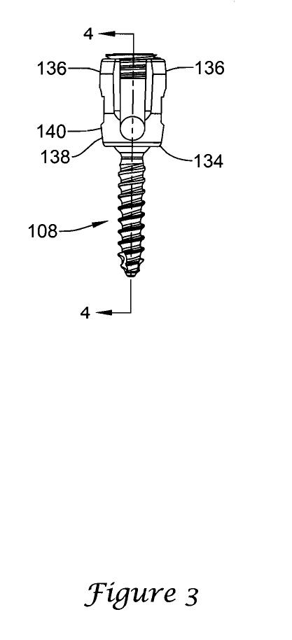

FIG. 4 is a cross-sectional view of the apparatus of FIG. 1 taken along line 4-4;

FIG. 5 is an exploded view of the apparatus of FIG. 1;

FIG. 6 is a perspective view of a spacer of the apparatus of FIG. 1;

FIG. 7 is a perspective view of a retaining member of the apparatus of FIG. 1;

FIG. 8A illustrates an implant apparatus with insufficient angularity for application through an access device; and

FIG. 8B illustrates an implant apparatus for which the angularity has been increased, e.g., by incorporating a biased angle design.

### DESCRIPTION

The illustrative embodiments described below relate to apparatuses for retaining bone portions, such as vertebrae of a spinal column, in a desired spatial relationship. In some embodiments, polyaxial screws and apparatuses comprising such screws, which may be used to retain bone portions in a desired spatial relationship, are provided. More particularly, biased or biased. angle polyaxial screws, which may achieve greater angularity between a housing and a fastener in some directions than in other directions can be provided. In some embodiments, the apparatuses may be oriented in order to achieve sufficient angularity to follow the curvature of the spine, especially in the cervicothoracic region. Also, the systems described herein enable a surgeon to perform a wide variety of methods as described herein. Some of the methods disclosed herein use an apparatus for retaining bone portions, such as vertebrae of a spinal column, in a desired spatial relationship. In some embodiments, methods of assembling an apparatus, e.g., of clamping a portion thereof, through a minimally invasive access device are provided. In some cases, apparatuses disclosed herein can be assembled without moving or without reorienting such an access device.

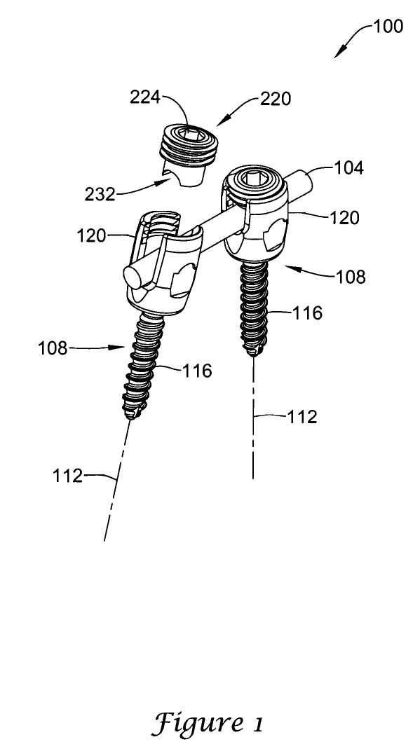

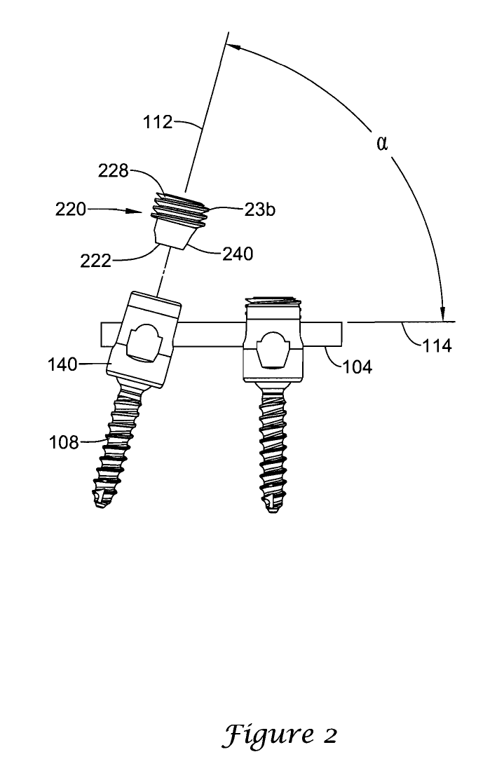

FIGS. 1-2 illustrate an apparatus 100 constructed according to one embodiment. The apparatus 100 can include longitudinal member or rod 104 that is configured to extend between portions of adjacent vertebrae, e.g., extending along the spinal column or spinous processes of the vertebrae. The longitudinal member 104 can be used to maintain or substantially maintain the spatial relationship of the adjacent bone portions. In some embodiments, the longitudinal member 104 is configured to preserve at least some of the normal motion of the portion of the patient's spine being treated. The longitudinal member 104 can be made of a suitable biocompatible material and can have a length that is at least sufficient to enable the member to span at least across a disc space between two adjacent vertebrae, e.g., between two adjacent pedicles. The length of the longitudinal member 104 can be selected based on the patient's needs and on the condition to be corrected, e.g., the number of vertebrae to be coupled together by the longitudinal member.

The longitudinal member 104 can be connected with vertebrae of the spinal column by fasteners 108 as discussed further below. The fastener 108 can be made of a suitable biocompatible material. The fastener 108 can have a longitudinal axis 112 and a threaded end portion 116 configured to engage the vertebra, e.g., in the vicinity of a pedicle.

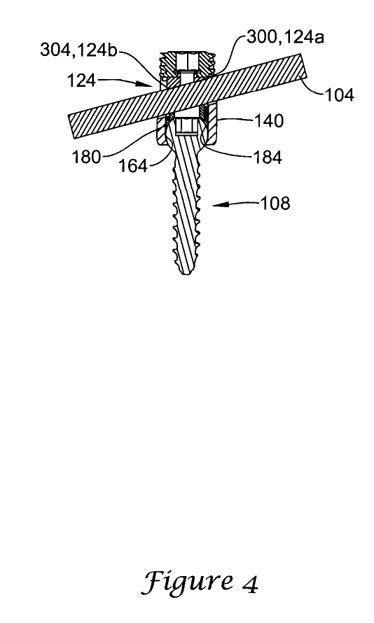

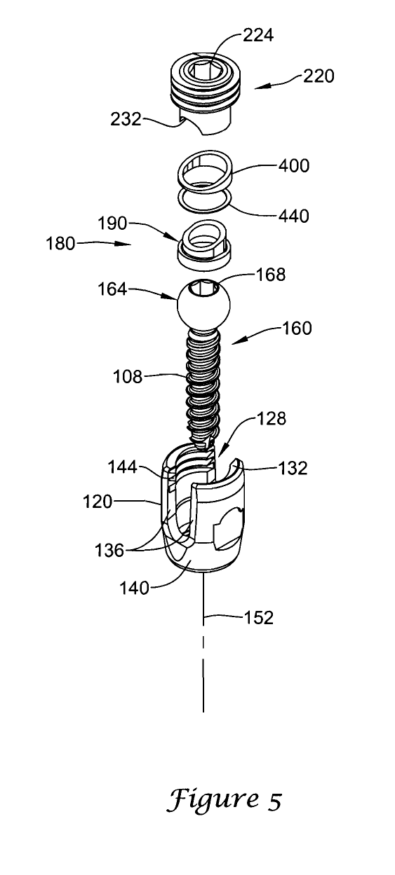

The fastener 108 preferably is extendable into a housing 120 that interconnects the longitudinal member 104 and the fastener 108. The housing 120 can include a first passage 124 through which the longitudinal member 104 can extend. See FIG. 4. The housing 120 can have a second passage 128 that extends generally transverse to the first passage 124. See FIG. 5. The fastener 108 is configured to extend through an opening 132 in the housing 120 and into the second passage 128. The second passage 128 is defined in part by a pair of part cylindrical members 136 that extend between the opening 132 and an opposite, fastener engaging end 140 of the housing 120. At least one and preferably both of the part cylindrical members 136 includes at least one thread 144. In one embodiment, the fastener engaging end 140 of the housing 120 has a cylindrical surface that is constricted 134 relative to the portion adjacent to the members 136. A tapered surface 138 can be provided extending from the part cylindrical members 136 to the constricted portion of the housing 120. As discussed further below, the tapered surface 138 and the constricted end 134 of the housing 120 together restrict or substantially prevent the fastener 108 from sliding out of the end of the housing 120 opposite the opening 132.

A second end portion 160 of the fastener 108 is provided with an enlarged head 164, which can include a spherical surface. A recess 168 can be provided on the second end portion 160 of the fastener 108. The recess 168 can be a hex-shaped or other suitable feature to facilitate driving the fastener 108 into a bone portion. In particular, the recess 168 can be configured to receive a tool that applies torque to the fastener 108 to turn the threads thereof into the vertebra. The enlarged head 164 of the fastener 108 can engage a tapered or constricted surface of the housing 120. Preferably such engagement enables the fastener 108 to be pivotable relative to the housing 120 so that the longitudinal axis 112 of the fastener 108 is positionable in any one of a plurality of angular positions relative to a longitudinal axis 152 of the passage 128.

FIGS. 4-6 illustrate embodiments in which a spacer 180 can be positioned in the second passage 128 of the housing 120. The spacer 180 has a lower portion 182 engageable with the fastener 108. A surface 184 of the lower portion 182 engages the enlarged head 164 of the fastener 108. In one arrangement, the surface 184 is a part spherical surface configured to engage a part spherical surface on the fastener 108. An axially extending portion 186 of the lower portion 182 extends from the surface 184 and is spaced from the enlarged head 164 of the fastener 108. The axially extending portion 186 helps position the spacer 180 in the housing 120.

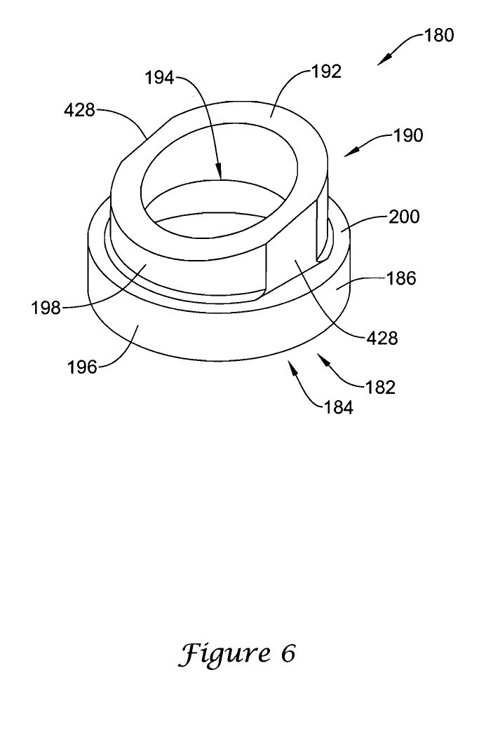

In some embodiments, the spacer 180 (FIG. 6) has an upper portion 190 with an upper surface 192 engageable with the longitudinal member 104. The spacer 180 has an axially extending opening 194 that extends through the upper portion 190 and the lower portion 182. A tool can be extended through the opening 194 to engage the recess 168 in the fastener 108. The tool extends through the opening 194 to apply torque to the fastener 108 to connect the fastener to the vertebra, as discussed above.

The lower portion 182 of the spacer 180 has a first outer surface 196, which can be cylindrical, with an outer size smaller than the passage 128. The upper portion 190 of the spacer 180 includes a second outer cylindrical surface 198 having a diameter smaller than the cylindrical surface 196. A radially extending surface 200 extends from the cylindrical surface 196 to the cylindrical surface 198. The radially extending surface 200 is a surface that extends generally transverse to the part cylindrical members 136. The radially extending surface 200 interacts with a member that enables the position of the housing 120 to be maintained relative to the position of the fastener 108, while maintaining the positionability thereof.

A clamping member or cap screw 220 is configured to threadably engage the threads 144 on the housing 120. The cap screw 220 engages, e.g., applies a force to the longitudinal member 104 to press the member 104 against the spacer 180. The spacer 180 is thereby pressed against the fastener 108. The cap screw 220 clamps the longitudinal member 104, the spacer 180, and the housing 120 to the fastener 108 to restrict, prevent or substantially reduce relative movement between the fastener, the housing and the member. Substantially reduce does not mean to completely eliminate because, for example, the longitudinal member 104 may be specifically configured to maintain some movement relative to the fastener 108. In other embodiments, the cap screw 220 may clamp only the longitudinal member 104 or the fastener 108 relative to the housing 120. One of skill in the art will also appreciate that it is not necessary to use a spacer 180 in some embodiments. In certain embodiments, the cap screw 220 or another suitable clamping mechanism will clamp at least one of the longitudinal member 104, the fastener 108, or the housing 120 to at least another of the longitudinal member, fastener, or housing without employing a spacer. Additionally, the fastener 108 and housing 120 may be integrally formed, such that only the longitudinal member 104 and housing 120 are clamped relative to one another.

The cap screw 220 or clamping member can be advanced into the threads 144 of the housing 120. A recess 224 can be provided in an end portion 228 of the cap screw 220 to facilitate such advancement. The recess 224 may have any suitable construction, such as being hex-shaped, and may be configured to receive a tool that applies torque to the cap screw 220 to engage the threads 144 of the upper portions 136 of the housing 120. The cap screw 220 can be advanced along an axis that is not orthogonal to the longitudinal member 104. The axis along which the cap screw 220 is advanced can correspond with the axis 152. Because the rod need not be straight but may be bent or curved, a reference location is defined near the engagement of the cap screw 220 with the longitudinal member 104, e.g., where the axis along which the cap screw is advanced intersects the longitudinal member 104. The axis along which the cap screw 220 is advanced is not orthogonal to the longitudinal member 104 at the reference location in some embodiments.

In some embodiments, the axis along which the clamping member 220 is advanced does not intersect the longitudinal member 104. One of skill in the art will appreciate that the clamping mechanism need not necessarily employ threads, as in a cap screw. The clamping mechanism could employ other mechanisms such as cambered flanges engaged in slots, so long as the longitudinal member 104 or like structure is relatively secure.

The bottom surface 222 of the cap screw 220 is configured to engage the longitudinal member 104 at an angle α less than ninety degrees relative to the axis along which the cap screw is advanced. FIG. 2 illustrates that the angle α can be an angle defined between the axis 112 and a longitudinal axis of the longitudinal member 104. The angle α also can be an angle between the axis 152 and the longitudinal axis 114 of the longitudinal member 104. The bottom surface 222 can be angled less than ninety degrees relative to the axis along which the cap screw 220 is advanced. In the embodiment illustrated in FIG. 5, the bottom surface 222 includes a hemi-cylindrical channel 232.

In one embodiment, the cap screw 220 includes a first or upper portion 236 that is configured to rotate relative to a second or lower portion 240 of the cap screw 220. The lower portion 240 includes members that define the sides of the channel 232 that can engage the rod 104 before the threads of the housing 120 and cap screw 220 have engaged. Such side members also can ensure proper alignment of the lower portion 240 relative to the longitudinal member 104. The engagement of the lower portion 240 with the longitudinal member 104 keeps the lower portion in the proper orientation such that the bottom surface 228 will be aligned with the longitudinal member 104 as these components engage each other. One of skill in the art will appreciate that the channel 232 of the cap screw 220 may be shaped in a variety of ways to facilitate engagement of the longitudinal member 104 at an angle not orthogonal to the axis along which the cap screw is advanced. The channel 232 can be of a shape other than hemi-cylindrical.

In some embodiments, the cap screw 220 is advanceable into engagement with the housing 120 along an axis. The axis of advancement of the cap screw 220 can be aligned with the axis 152 or another axis of the passage 128. The axis of advancement of the cap screw can be aligned with the axis 112 in some cases. The cap screw 220 is configured to engage the longitudinal member 104 at a first location 300 and a second location 304, as shown in FIG. 4. The first location 300 is adjacent a first end 124A of the passage 124 of the housing 120 and the second location 304 is adjacent a second end 124B of the passage 124 in one arrangement. The first location 300 can be higher than the second location 304 in a direction along the axis of advancement of the cap screw 220. As used in this context, “higher” means that the first location 300 is spaced farther from the point of engagement of the cap screw 220 with the longitudinal member 104 than is the second location 304. Stated another way, a projection of the second location 304 onto the axis of advancement of the clamp screw 220 is between a projection of the first location 300 onto the axis of advancement of the cap screw and an intersection of the axis of advancement and the longitudinal member 104.

In the embodiment illustrated in FIGS. 4-6, the upper surface 192 of the spacer 180 is configured to engage the longitudinal member 104. In certain embodiments, the upper surface 192 is angled relative to a plane normal to a longitudinal axis of the spacer 180. In this context, the longitudinal axis of the spacer is a central axis thereof that is aligned or parallel with the axis of advancement of the cap screw 220 when the screw is applied to the housing 120, e.g., the central axis of the opening 194. The upper surface 192 of the spacer 180 may be substantially flat or of any shape suitable to engage the longitudinal member 104 at the desired angle. Preferably, the space formed between the spacer 180 and the cap screw 220 or clamping mechanism is configured such that the longitudinal member 104 will be gripped at an angle not orthogonal to the axis along which the clamping mechanism is advanced.

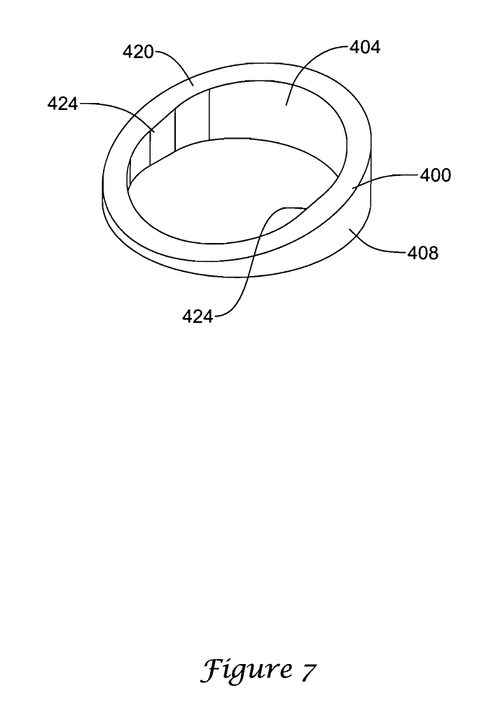

FIGS. 5 and 7 show a ring-shaped positioning or retaining member 400 that holds the spacer 180 in the housing 120. The retaining member 400 has an inner cylindrical surface 404 with a diameter slightly larger than the outside diameter of the outer cylindrical surface 198 on the spacer 180. The retaining member 400 has a outer cylindrical surface 408 that engages the housing 120. The outer cylindrical surface 408 is sized to fit into the upper portion of the housing 120, but is slightly larger than the diameter of second or lower cylindrical surface 140 of the housing. Accordingly, the retaining member 400 can be easily inserted into the housing 120. As it is being inserted, the retaining member 400 engages the tapered surface 138 of the housing 120. The retaining member 400 can thereafter be press fit into engagement with an inner surface of the housing 120. In another embodiment, the retaining member 400 can be connected to the housing 120 by one or more, e.g., a pair of diametrically opposed, circumferential welds.

FIGS. 5 and 7 illustrate that the in one embodiment, the retaining member 400 has an upper surface 420 that is tilted at an angle, which can be the same angle as the upper surface 192 of the spacer 180. The retaining member 400 also can have one or more, e.g., two diametrically opposed, flat surfaces 424 on an inner surface thereof configured to restrict or substantially prevent axial rotation of the spacer 180 relative to the retaining member 400. The spacer 180 can have similar flat surfaces 428 configured to engage the flat surfaces 424 of the retaining member 400. Though flat surfaces are shown in one illustrative embodiment, other anti-rotation features could be substituted. In certain embodiments in which a retaining member 400 is not present, anti-rotation features similar to the flat surfaces 428 on the spacer 180 may interact with flat surfaces on the housing (not shown) to restrict or substantially prevent rotation but allow axial movement of the spacer 180 relative to the housing 120.

A structure can be provided to urge the spacer 180 into engagement with the fastener 108. For example, a ring-shaped spring member 440 can be provided between the retaining member 400 and the spacer 180. See FIG. 5. The spring member 440 engages the spacer 180 to apply an axial force to the spacer to restrict or substantially prevent relative movement between the fastener 108 and the housing 120 when the rod 104 is disengaged from the spacer. More particularly, the spring member 440 urges the spacer 180 axially to generate or increase a frictional engagement between the fastener and the spacer. The fastener 108 and the housing 120 are manually movable relative to each other by a surgeon when the rod 104 is disengaged from the spacer 180 and the spring member 440 applies the axial force.

The spring member 440 has a suitable shape or configuration, such as an arched or wavy shaped when the spring member is disengaged from the spacer 180 and the retaining member 400. When the spring member 440 is received between the spacer 180 and the retaining member 400, the spring member is compressed and applies an axial force to the spacer.

The apparatus 100 is particularly well suited for minimally invasive procedures. In one such procedure, the apparatus 100 is applied to the spine through an access device or a retractor, such as described in the attached appendix and in U.S. application Ser. No. 11/490,511 (filed Jul. 20, 2006 published Jan. 25, 2007 as Publication No. U.S. 2007/0021750A1), U.S. Pat. No. 7,144,396, and in PCT Publication No. WO 2006/045089 published Apr. 27, 2006, each of which is hereby incorporated by reference in their entirety and should be considered a part of this specification.

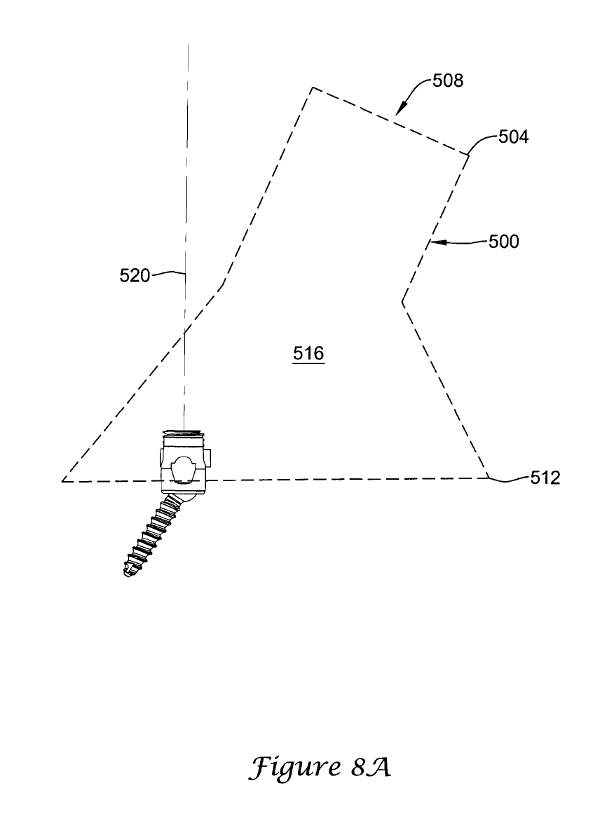

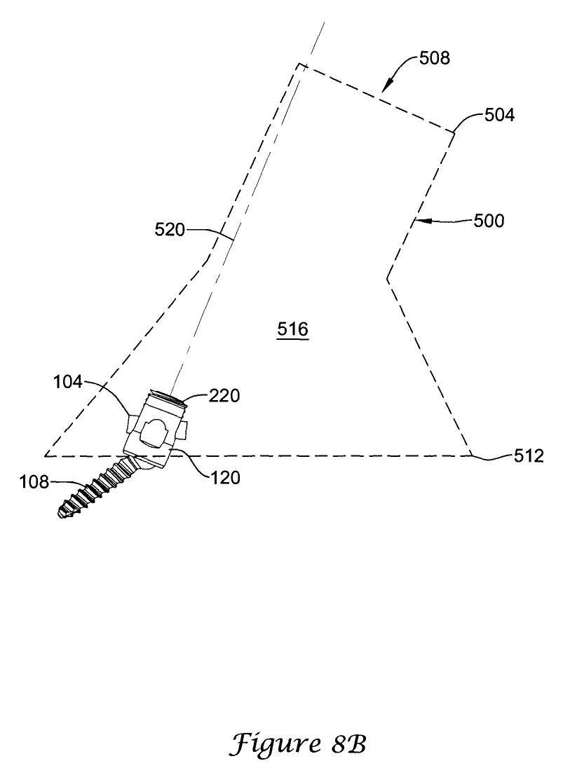

A preliminary step in such a procedure is to deliver an access device 500 to a location adjacent the spine. The access device 500 is shown schematically in FIGS. 8A and 8B. In various techniques, the location of insertion may be a lumbar, thoracic or cervical portion of the spine. At least a portion of the access device optionally is expanded to increase access to a surgical location. In the embodiment shown in FIGS. 8A and 8B, the distal end is expanded. In a one level fixation procedure, the access device provides access to two adjacent vertebrae, e.g., the pedicles or lateral masses of two adjacent vertebrae. Additional adjacent vertebrae may be exposed by the access device for procedures performed over longer surgical fields, such as across three or more adjacent vertebrae. The apparatus 100 is inserted through the access device 500.

Thereafter, a tool is inserted through the opening 194 in the spacer 180 and into the recess 168 in the fastener 108. The fastener 108 preferably is advanced through the access device 500 to the surgical locations. Torque is applied to the fastener 108 to advance the fastener 108 into the vertebra. Once the fastener 108 is connected with the vertebra, the housing 120 can be positioned relative to the fastener. The spring member 440 maintains the position of the housing 120 relative to the fastener 108 when the rod 104 is disengaged from the spacer 180. By enabling the housing 120 to be maintained in a selected position relative to the fastener 108, the surgeon's hands are free to manipulate other tools or implants to complete the procedure. This feature simplifies and shortens the procedure, benefiting the patient and the surgeon.

Once the housing 120 is positioned relative to the fastener 108, the rod 104 is placed into the passage 124 and in engagement with the spacer 180. Placing the rod 104 in the passage 124 may be facilitated by a suitable tool, such as a grasper apparatus. Also, placing the rod 104 may include additional optional steps to manipulate vertebrae, such as a spondy reduction procedure. Spondy procedures and tools configured to perform them are described in U.S. Pat. No. 6,648,888 and PCT Application No. PCT/US03/27879 (filed Sep. 5, 2003 and PCT Publication WO 04/022 128 published Mar. 18, 2004), which are hereby expressly incorporated by reference herein in their entirety.

The cap screw 220 is threaded into the housing 120 and into engagement with the rod 104. A screwdriver apparatus may be used to thread the cap screw 220 into the housing 140. The cap screw 220 clamps the rod 104, the spacer 180, and the housing 120 to the fastener 108 to restrict or substantially prevent movement of the fastener relative to the housing. Alternatively, the fastener 108 can be connected to the vertebra prior to the spacer 180, the spring member 440, and the retaining member 400 being inserted into the housing 120.

If the apparatus 100 is deployed in a minimally invasive procedure, delivery of the cap screw 220 may be facilitated by a guide apparatus or other similar tool. Prior to clamping the cap screw 220, additional procedures that manipulate the position of the screw 108 relative to another screw 108 or the position of adjacent vertebrae may be performed. Such procedures include compression and distraction procedures, as described in U.S. Pat. No. 7,004,947 and PCT Application No. PCT/US03/020003 (filed Jun. 24, 2003 and PCT Publication WO 04/000145 published Dec. 31, 2003), which are hereby expressly incorporated by reference herein in their entirety.

In one arrangement, a spherical surface of the enlarged head 164 of the fastener 108 engages a corresponding (e.g., spherical) surface in the second passage of the housing 120. This arrangement enables the fastener 108 to be universally pivotable relative to the housing 120 so that the longitudinal axis 112 of the fastener 108 is positionable in any one of a plurality of angular positions relative to the longitudinal axis 152 of the passage 128.

The range of angular positions provided by the apparatus 100 is generally not uniform about the axis 112 of the fastener 108. For example, in one embodiment, the housing 120 can be held at a larger angle on one side of the fastener 108 than on another side of the fastener. This biased angularity can be accomplished through the non-orthogonality of the rod 104 and the axis along which the cap screw 220 or other clamping mechanism is advanced. In some embodiments, the angle between the axis of advancement of the clamping mechanism and a longitudinal axis of the longitudinal member at a reference location is 85 degrees or less. In other embodiments, the fastener 108 can achieve thirty degrees more angularity or angulation in one direction along the rod than in the opposite direction along the rod. The ability of an assembly comprising multiple apparatuses 100 coupled with a longitudinal member 104 to conform to varied anatomy advantageously improves as higher degrees of angularity are possible. In particular, in certain regions of the spine, greater curvature is present than in other regions. The cervical region exhibits greater curvature than the lumbar region. By providing greater angularity or angulation in one direction, than in another, the apparatus 100 can provide a greater angle for the housing 120 relative to the fastener 108. This arrangement enables the fastener 108 to point to a greater degree toward the head of the patient while enabling the housing 120 to be oriented toward a proximal end opening of the access device 500, as discussed in more detail below.

Referring to FIGS. 8A and 8B, the benefits of greater angularity will be discussed in greater detail. As discussed above, some spinal procedures can be performed through an access device 500. The access device 500 can have a proximal end 504 that defines an opening 508 into which the apparatus 100 and related surgical instruments and implants can be inserted to a spinal location being treated. The access device 500 also has a distal end 512 that can be disposed near the spinal region to be treated and an access path 516 can be defined between the proximal and distal ends 504, 512 such that insertion of these implants and instruments can be facilitated. One advantageous access device 500 has a distal end 512 that is larger than the proximal end 504. While such an access device is advantageous in that it limits tissue disruption, the opening is not directly above all regions of the distal end 512. While the access device 500 can be manipulated to try to align the proximal end with the distal end, the curvature of the spine and the configuration of spinal screws with insufficient angularity may not allow sufficient access to perform the procedure.

For example, a screw with symmetrical angularity, e.g., equal amounts of tilt of a housing relative to a fastener, may not have enough angularity at either extremes of the tilt such that when fully tilted, an access trajectory 520 along which a tool is to be inserted to access the fastener, e.g., to deliver a cap screw, may intersect the access device 500. In particular, rather than extending through the opening 508 defined at the proximal end 504, the access trajectory 520 would extend through a side of the device. This would block access to the portion of the fastener being accessed, preventing, for example, implantation of a fastener or advancement of a clamp screw. See FIG. 8A.

In contrast, a biased angle arrangement such as described above permits a housing to tilt more to one side of the axis of the fastener than to another. By biasing the tilt angle to one side, the largest tilt angle is increased. As such, a large angle of entry of a cap screw 220 can be achieved, enabling the access trajectory 520 of the instrument to pass through the opening 508 of the proximal end 504 of the device 500. See FIG. 8B.

The various devices, methods and techniques described above provide a number of ways to carry out the invention. Also, although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of the illustrative embodiments herein.

Many of the systems, apparatuses, methods, and features described herein can be combined with many of the systems, apparatuses, methods and features disclosed in the following patents and patent applications. The entire disclosure of all of the following patents and patent applications is hereby incorporated by reference herein and made a part of this specification: U.S. Pat. No. 6,361,488 (issued Mar. 26, 2002), U.S. Pat. No. 6,530,880 (issued Mar. 11, 2003), U.S. Pat. No. 6,648,888 (issued Nov. 18, 2003), U.S. Pat. No. 6,652,553 (issued Nov. 25, 2003), U.S. Pat. No. 6,641,583 (issued Nov. 4, 2003), U.S. Pat. No. 6,554,832 (issued Apr. 29, 2003), U.S. Pat. No. 6,673,074 (issued Jan. 6, 2004), U.S. Pat. No. 6,641,583 (issued Nov. 4, 2003), U.S. Pat. No. 6,554,832 (issued Apr. 29, 2003), U.S. Pat. No. 6,673,074 (issued Jan. 6, 2004), U.S. Pat. No. 6,821,243 (issued Nov. 23, 2004), U.S. Pat. No. 6,837,889 (issued Jan. 4, 2005), U.S. Pat. No. 7,056,321 (issued Jun. 6, 2006), U.S. patent application Ser. No. 10/075,668 (filed Feb. 13, 2002, published Aug. 14, 2003 as Publication No. U.S. 2003/0153911), Ser. No. 10/178,875 (filed Jun. 24, 2002, published Dec. 25, 2003 as Publication No. U.S. 2003/0236529), Ser. No. 10/280,799 (filed Oct. 25, 2002), Ser. No. 10/361,887 (filed Feb. 10, 2003, published Aug. 14, 2003 as Publication No. U.S. 2003/0153927), Ser. No. 10/969,788 (filed Oct. 20, 2004, published Aug. 4, 2005 as Publication No. U.S. 2005/0171551), Ser. No. 10/483,605 (published Sep. 9, 2004 as Publication No. 2004/0176766), Ser. No. 10/658,736 (filed Sep. 9, 2003, published Jul. 8, 2004 as Publication No. U.S. 2004/0133201), Ser. No. 10/678,744 (filed Oct. 2, 2003, published Apr. 7, 2005 as Publication No. U.S. 2005/0075540), Ser. No. 10/693,815 (filed Oct. 24, 2003, published Apr. 28, 2005 as Publication No. U.S. 2005/0090822), Ser. No. 10/693,250 (filed Oct. 24, 2003, published on Apr. 28, 2005 as Publication No. U.S. 2005/0090899), Ser. No. 10/693,663 (filed Oct. 24, 2003, published on Apr. 28, 2005 as Publication No. U.S. 2005/0090833), Ser. No. 10/842,651 (filed May 10, 2004, published on Apr. 7, 2005 as Publication No. U.S. 2005/0075644), Ser. No. 10/845,389 (filed May 13, 2004, published on Nov. 18, 2004 as Publication No. U.S. 2004/0230100), Ser. No. 10/969,293 (filed Oct. 20, 2004, published on Apr. 20, 2006 as Publication No. U.S. 2006/0084981), Ser. No. 11/094,822 (filed Mar. 30, 2005, published on Nov. 10, 2005 as Publication No. U.S. 2005/0251192), Ser. No. 10/926,579 (filed Aug. 26, 2004, published Dec. 8, 2005 as Publication No. U.S. 2005/0273131), Ser. No. 10/926,840 (filed Aug. 26, 2004, published Dec. 8, 2005 as Publication No. U.S. 2005/0273132), Ser. No. 10/927,633 (filed Aug. 26, 2004, published Dec. 8, 2005 as Publication No. U.S. 2005/0273133), Ser. No. 10/969,124 (filed Oct. 20, 2004, published May 19, 2005 as Publication No. U.S. 2005/0107789), Ser. No. 10/972,987 (filed Oct. 25, 2004, published Nov. 3, 2005 as Publication No. U.S. 2005/0245942), Ser. No. 11/241,811 (filed Sep. 30, 2005, published Mar. 30, 2006 as Publication No. U.S. 2006/0069404), Ser. No. 11/238,109 (filed Sep. 27, 2005), Ser. No. 11/238,109 (filed Sep. 27, 2005), U.S. Provisional Applications No. 60/471,431 (filed May 16, 2003), 60/497,763 (filed Aug. 26, 2003), 60/497,822 (filed Aug. 26, 2003), 60/513,796 (filed Oct. 22, 2003), 60/513,013 (filed Oct. 23, 2003), 60/514,559 (filed Oct. 24, 2003), 60/545,587 (filed Feb. 18, 2004), 60/558,296 (filed Mar. 31, 2004), 60/579,643 (filed Jun. 15, 2004), and 60/625,782 (filed Nov. 5, 2004).

What is claimed is:

1. An apparatus for connecting a longitudinal member to a bone portion comprising:
a fastener having first and second ends and a longitudinal axis, said first end engageable with a bone portion;
a housing having a first passage configured to receive a longitudinal member, and a second passage configured to receive said fastener, said fastener extending through an opening in said housing into said second passage and being movable relative to said housing, said longitudinal axis of said fastener being positionable in any one of a plurality of angular positions relative to a longitudinal axis of said second passage;
a clamping member configured to be received in threaded engagement within said housing, said clamping member having a first end configured such that, when a longitudinal member is positioned in said first passage, said first end of said clamping member engages said longitudinal member at an angle not orthogonal to an axis along which the clamping member is advanced.

2. The apparatus of claim 1, wherein said angle is defined between said fastener axis and a longitudinal axis of the longitudinal member.

3. The apparatus of claim 1, wherein said angle is defined between said second passage axis and a longitudinal axis of the longitudinal member.

4. The apparatus of claim 3, wherein said angle is less than 90 degrees.

5. The apparatus of claim 1, wherein said first end of said clamping member includes a channel configured to engage the longitudinal member.

6. The apparatus of claim 5, wherein the clamping member is configured to engage said longitudinal member at first and second locations, wherein said first location is higher than said second location in a direction along the axis of advancement of the clamping member.

7. The apparatus of claim 1, wherein said clamping member has a second end including a recess configured to receive a tool for applying torque to the clamping member.

8. The apparatus of claim 1, wherein said clamping member has external threading that matches internal threading on an interior of said housing.

9. The apparatus of claim 1, further including a spacer configured to be received in said second passage of said housing, said spacer having a first end engageable with said fastener and a second end engageable with said longitudinal member.

10. An apparatus for connecting a longitudinal member to a bone portion comprising:
a fastener having a threaded shank for engaging a bone portion and an enlarged head;
a housing having a first passage and a second passage having a longitudinal axis extending transverse to the first passage, said first passage configured to receive a longitudinal member, said second passage configured to receive said fastener, said fastener extending through an opening in said housing into said second passage and being movable relative to said housing, said longitudinal axis of said fastener being positionable in any one of a plurality of angular positions relative to the longitudinal axis of said second passage;
a spacer configured to be received in said second passage, said spacer having a top surface configured to engage said longitudinal member at an angle that is not orthogonal to a longitudinal axis of said longitudinal member; and
a clamping member configured to be received in threaded engagement within said housing, wherein a first end of said clamping member includes a surface angled less than 90 degrees relative to an axis along which the clamping member is advanced.

11. The apparatus of claim 10, wherein the housing has one or more flat surfaces within the second passage and the spacer has one or more external flat surfaces that engage the one or more flat surfaces within the second passage of the housing.

12. An apparatus for connecting a longitudinal member to a bone portion comprising:
a fastener having first and second ends and a longitudinal axis, said first end engageable with a bone portion;
a longitudinal member;
a housing having a first passage configured to receive said longitudinal member, and a second passage configured to receive said fastener, said fastener extending through an opening in said housing into said second passage and being movable relative to said housing, said longitudinal axis of said fastener being positionable in any one of a plurality of angular positions relative to a longitudinal axis of said second passage;
a clamping member configured to be received in threaded engagement within said housing, said clamping member having a first end configured such that, when said longitudinal member is positioned in said first passage, said first end of said clamping member engages said longitudinal member at an angle not orthogonal to an axis along which the clamping member is advanced.

13. The apparatus of claim 12, wherein a first end of said clamping member includes a surface angled less than 90 degrees relative to the axis along which the clamping member is advanced.

14. The apparatus of claim 12, further including a spacer configured to be received in said second passage of said housing, said spacer having a first end engageable with said fastener and a second end engageable with said longitudinal member.

15. An apparatus for connecting a longitudinal member to a bone portion comprising:
a fastener having a threaded shank for engaging a bone portion and an enlarged head;
a housing having a first passage and a second passage having a longitudinal axis extending transverse to the first passage, said first passage configured to receive a longitudinal member, said second passage configured to receive said fastener, said fastener extending through an opening in said housing into said second passage and being movable relative to said housing, said longitudinal axis of said fastener being positionable in any one of a plurality of angular positions relative to the longitudinal axis of said second passage;
a spacer configured to be received in said second passage, said spacer having a top surface angled to engage said longitudinal member at an angle that is not orthogonal to a longitudinal axis of said longitudinal member;
a retaining member having an inner dimension sized to fit over at least a portion of said spacer and an outer dimension sized to engage an inner surface of said housing; and
a clamping member configured to be received in threaded engagement within said housing, wherein a first end of said clamping member includes a surface angled less than 90 degrees relative to an axis along which the clamping member is advanced.

16. The apparatus of claim 15, said housing having an upper portion and a lower portion, the lower portion being constricted relative to the upper portion, wherein the constricted lower portion is configured to retain the enlarged head of the fastener.

17. The apparatus of claim 16, wherein the outer dimension of said retaining member is sized to fit within the upper portion of said housing, but is larger than an internal dimension of the lower portion of said housing.

18. The apparatus of claim 15, wherein said retaining member has an upper surface angled to match the top surface of said spacer.

19. The apparatus of claim 15, further comprising a spring member configured to apply an axial force on said spacer to restrict relative movement between the fastener and housing.

20. The apparatus of claim 15, wherein said first end of said clamping member includes a channel configured to engage the longitudinal member.

21. A method of securing vertebrae at a surgical site in a patient, said method comprising:
inserting an access device into the patient to a surgical site adjacent the spine, the access device having a distal end larger than a proximal end, and an access path therebetween;
inserting a first fixation device through the access device;
securing the first fixation device to a first vertebra, the first fixation device including a first fastener and a first housing, the first housing having a first passage configured to receive a longitudinal member and a second passage configured to receive the first fastener, the first fastener extending through an opening in the first housing and being universally pivotable relative to said first housing, wherein said securing step includes positioning said first housing such that a longitudinal axis of the first fastener is in any one of a plurality of angular positions relative to a longitudinal axis of said second passage of said first housing;
inserting a longitudinal member through the access device and into said first housing; and
inserting a first clamping member into said first housing, said first clamping member having a first end configured to engage said longitudinal member at a first angle, wherein said first angle is not orthogonal to an axis along which the first clamping member is advanced;
wherein said first housing is positioned such that an access trajectory along which said first clamping member is inserted passes through said access device.

22. The method of claim 21, wherein after said first fixation device is inserted, the method further comprises the step of inserting a first spacer into said first housing, said first spacer having a first end configured to engage said longitudinal member at an angle that is not orthogonal to a longitudinal axis of said longitudinal member.

23. The method of claim 21, wherein after said securing step, the method further comprises:
inserting a second fixation device through the access device; and
securing the second fixation device to a second vertebra, the second fixation device including a second fastener and a second housing, the second housing having a first passage configured to receive a longitudinal member and a second passage configured to receive the second fastener, the second fastener extending through an opening in the second housing and being universally pivotable relative to said second housing, wherein said securing step includes positioning said second housing such that a longitudinal axis of the second fastener is in any one of a plurality of angular positions relative to a longitudinal axis of said second passage of said second housing;
wherein said longitudinal member is inserted into said first and second housings, said method further comprising inserting a second clamping member into said second housing, said second clamping member having a first end configured to engage said longitudinal member at a second angle.

24. The method of claim 23, wherein the first and second angles along which said first and second clamping members are advanced are different.

25. The method of claim 23, wherein after said second fixation device is inserted, the method further comprises the step of inserting first and second spacers into said first and second housings, said first and second spacers each having a first end configured to engage said longitudinal member at an angle that is not orthogonal to a longitudinal axis of said longitudinal member.

26. The method of claim 25, wherein the angles at which said first ends of said first and second spacers engage said longitudinal member are different.
